(19) United States
(12) Patent Application Publication
(10) Pub. No.: US 2019/0125821 A1
(43) Pub. Date: May 2, 2019

(54) COMPOSITION FOR TREATING AND PREVENTING RHEUMATOID ARTHRITIS

(71) Applicants: Joshua M. COSTIN, Naples, FL (US); John M WILLIAMS, Bonita Springs, FL (US); HSRX GROUP, LLC, Tucson, AZ (US)

(72) Inventors: Joshua M COSTIN, Naples, FL (US); John M. WILLIAMS, Bonita Springs, FL (US); Dan LI, Harrington Park (SG)

(21) Appl. No.: 16/083,329

(22) PCT Filed: Mar. 8, 2017
(86) PCT No.: PCT/IB17/51365

§ 371 Date: Sep. 7, 2018

Related U.S. Application Data
(60) Provisional application No. 62/305,072, filed on Mar. 8, 2016.
(60) Provisional application No. 62/415,713, filed on Nov. 1, 2016.

(51) Int. Cl. A61K 36/9066; A61K 31/519; A61K 39/395; A61K 9/00; A61P 19/02; A61P 29/00

(52) U.S. Cl. CPC A61K 36/9066; A61K 31/519; A61K 39/3955; A61K 9/0053; A61P 29/00; A61K 9/0014; A61K 9/0043; A61P 19/02; A61K 9/0019

(57) ABSTRACT

The present invention relates generally to compositions and methods of use that include compounds that treat and prevent rheumatoid arthritis, inflammation, and diseases caused by or having inflammation as a symptom.

### CROSS-REFERENCE TO RELATED APPLICATIONS

This application claims the benefit of U.S. Provisional Application No. 62/305,072, filed on Mar. 8, 2016 and U.S. Provisional Application No. 62/415,713, filed on Nov. 1, 2016, the contents of which are incorporated into the present application by reference.

### BACKGROUND OF THE INVENTION

### Field of the Invention

The present invention relates to formulations containing a mixture of compounds capable of preventing and treating rheumatoid arthritis and/or inflammation.

### Description of Related Art

Rheumatoid arthritis (RA) is an autoimmune disorder that causes chronic inflammation of the synovium, the lining of the membrane that surrounds joints (Mayo Clinic, Rheumatoid arthritis, 2014). The inflammation in RA causes swelling that can result in bone erosion, joint deformity, and pain. Id. RA may also affect other parts of the body, such as the eyes, lungs, blood vessels, and skin and eventually lead to osteoporosis, carpal tunnel syndrome, hardened and blocked arteries, inflammation of the sac that encloses the heart, and inflammation and scarring of lung tissue. Id. Some symptoms of RA include fatigue, fever, weight loss, bumps of tissue under the skin of the arms, stiffness in the joints in the morning that may last for hours, and joints that are tender, warm, and swollen. Id.

No cure currently exists for RA, but anti-inflammatory drugs, such as nonsteroidal anti-inflammatory drugs (NSAIDs) and steroids, may be used to reduce inflammation and relieve pain (Mayo Clinic, Rheumatoid arthritis, 2014). Further, disease-modifying antirheumatic drugs (DMARDs) and a new classes of biological agent DMARDs (“biologics”) can be used to slow the progression of rheumatoid arthritis.

Inflammation is an underlying factor in rheumatoid arthritis as well as other diseases such as polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. There are multiple proteins involved in inflammation including: cyclooxygenases COX1, COX2, 5LOX; cytokines such as IL-6 and TNF-α; and cytokines that are produced in T-helper 1 (Th16) responses such as IFNγ. In particular, TNF-α and IL-6 are cytokines that are abundant in patients with rheumatoid arthritis (Gottenberg et al., 2012; Hennigan et al., 2008).

Inhibition of inflammation is a method used to combat the causes and/or symptoms of RA and other inflammatory diseases. Antibodies against TNF-α and IL-6 receptors, Adalimumab (Humira, Abbvie, USA) and Tocilizumab (Roactemra, Roche, USA) respectively, have been shown to be efficacious for reduction in symptoms among some, but not all, patients with rheumatoid arthritis (Kremer et al., 2011; Nishimoto et al., 2014; Nishimoto et al., 2006). Adalimumab has also been shown to be effective for some patients having polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. However, use of these antibodies have significant side effects associated with their use, including redness, itching, pain, bruising, or swelling at the injection site, headache, stuffy nose, sinus pain, or stomach pain. There is additionally an increased risk of serious infections like tuberculosis and sepsis that can lead to hospitalization and/or death.

Some prostaglandins (PG) are also involved in inflammation. Prostaglandins are lipid molecules that have physiological hormone like effects. Among the various PG species, two prostanoids, PGI-2 and PGE-2, have both been implicated as the most responsible species in inflammation because of their abundance in inflammatory exudates and tissues (Huwiler et al., 2009; Park et al., 2006; Qin et al., 2014; Tsai et al., 2014). Cyclooxygenases mediate the production of these prostanoids as end products of arachidonic acid metabolism. PGE-2 is a principal mediator of inflammation in diseases such as rheumatoid arthritis (Choi et al., 2014; Huwiler et al., 2009; Wei Zuo et al., 2011). PGE-2 signaling is mediated by interactions with four distinct G protein-coupled receptors, E-prostanoid (EP) receptors, EP1-4, and potentially antagonistic signaling cascades. Activation by PGE-2 leads to changes in the production of cAMP and/or phosphoinositol turnover and intracellular Ca²⁺ mobilization (Andreasson, 2010).

Inhibition of PGE-2 synthesis has been an important anti-inflammatory strategy for treatment for more than 100 years. Pharmacologic PGE-2 blockage with aspirin and later NSAIDs has been a useful anti-inflammatory strategy for more than a century, but the degree and severity of gastrotoxicity with chronic NSAIDs use became apparent more recently (Park et al., 2006).

### SUMMARY OF THE INVENTION

The present invention provides a solution to the current problems facing treatment and prevention of rheumatoid arthritis and/or inflammation. The inventors have surprisingly determined that a combination of several compounds found in turmeric can prevent and treat rheumatoid arthritis and inflammation. The inventors have also determined that specific relative concentrations of the compounds enhance the ability of the combined compounds to prevent and treat rheumatoid arthritis and inflammation. In addition, the inventors have determined that using compounds of the present invention with additional agents for treating or preventing rheumatoid arthritis and inflammation enhance the ability of the combined compounds to prevent and treat rheumatoid arthritis and inflammation.

The inventors have also surprisingly determined that the compounds and compositions disclosed herein can prevent and treat the following diseases: polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. Without wishing to be bound by theory, it is believed that at least some of the mechanisms of action of the compounds and compositions disclosed herein include those that address the underlying causes or symptoms of these diseases. Further, it is expected that using the compounds and compositions of the present invention with additional dugs will enhance the ability of the combined compounds to prevent and treat these diseases.

In one aspect, disclosed is a composition of any one of, any combination of, or all of twenty-two biomarkers. In one instance the composition includes any one of, or any combination of, or all of the following biomarkers: biomarker 11 having an accurate mass of 232.146 amu and having a relative abundance of at least 2.53%; biomarker 1 having an accurate mass of 146.113 amu and having a relative abundance of at least 0.20%; biomarker 2 having an accurate mass of 160.116 amu and having a relative abundance of at least 0.51%; biomarker 3 having an accurate mass of 176.128 amu and having a relative abundance of at least 0.35%; biomarker 4 having an accurate mass of 178.129 amu and having a relative abundance of at least 0.30%; biomarker 5 having an accurate mass of 180.106 amu and having a relative abundance of at least 0.10%; biomarker 6 having an accurate mass of 194.131 amu and having a relative abundance of at least 0.21%; biomarker 7 having an accurate mass of 198.146 amu and having a relative abundance of at least 2.86%; biomarker 8 having an accurate mass of 204.188 amu and having a relative abundance of at least 4.51%; biomarker 9 having an accurate mass of 218.167 amu and having a relative abundance of at least 88.89%; biomarker 10 having an accurate mass of 220.178 amu and having a relative abundance of at least 5.15%; biomarker 12 having an accurate mass of 234.166 amu and having a relative abundance of at least 8.04%; biomarker 13 having an accurate mass of 236.177 amu and having a relative abundance of at least 0.80%; biomarker 14 having an accurate mass of 238.191 amu and having a relative abundance of at least 0.13%; biomarker 15 having an accurate mass of 248.145 amu and having a relative abundance of at least 0.54%; biomarker 16 having an accurate mass of 268.189 amu and having a relative abundance of at least 0.18%; biomarker 17 having an accurate mass of 316.209 amu and having a relative abundance of at least 0.20%; biomarker 18 having an accurate mass of 326.234 amu and having a relative abundance of at least 0.28%; biomarker 19 having an accurate mass of 334.212 amu and having a relative abundance of at least 0.16%; biomarker 20 having an accurate mass of 350.230 amu and having a relative abundance of at least 0.21%; biomarker 21 having an accurate mass of 436.338 amu and having a relative abundance of at least 0.90%; wherein the biomarkers are found in Curcuma longa, and wherein the relative abundance is relative to 25 mg/ml salicylic acid spiked in 500 ng/ml of the composition. In some instances, the biomarkers contained in the composition disclosed above have a relative abundance of at most: biomarker 11 of 4.70%; biomarker 1 of 0.37%; biomarker 2 of 0.94%; biomarker 3 of 0.65%; biomarker 4 of 0.55%; biomarker 5 of 0.19%; biomarker 6 of 0.39%; biomarker 7 of 5.32%; biomarker 8 of 8.38%; biomarker 9 of 165.08%; biomarker 10 of 9.56%; biomarker 12 of 14.94%; biomarker 13 of 1.49%; biomarker 14 of 0.25%; biomarker 15 of 1.01%; biomarker 16 of 0.33%; biomarker 17 of 0.38%; biomarker 18 of 0.52%; biomarker 19 of 0.30%; biomarker 20 of 0.39%; and biomarker 21 of 1.66%; wherein the relative abundance is relative to 25 mg/ml salicylic acid spiked in 500 mg/ml of the composition. In some instances, the composition further includes biomarker 22, having an accurate mass of 216.151 amu. In some instances, biomarker 22 is present in the composition at at least 5.54 μg/ml. In some instances, any one of the compositions disclosed herein contains at most 10.29 μg/m1 of biomarker 22. In some instances, any one of the compositions disclosed herein contains at least 2, 3, 4, 5, 6, 7, 8, 9, 10, 11, 12, 13, 14, 15, 16, 17, 18, 19, 20, 21, or 22 of biomarkers 1 through 22. In some instances, the mass of each biomarker is the mass as determined by a Direct Analysis in Real Time-TOF (DART-TOF) mass spectrometer.

In some aspects, any one of the compositions disclosed above contains at least one biomarker that is synthetically obtained. In some aspects, any one of the compositions disclosed above contains at least one biomarker that is isolated from a plant. In some instances, the plant is Curcuma longa. In some aspects, any one of the compositions disclosed above has an at least 90%, preferably at least 95%, or at least 98% batch-to-batch chemical consistency of relative abundance for the biomarkers.

In some aspects, any one of the compositions disclosed herein further contains a preservative. In some aspects, any one of the compositions disclosed herein further contains at least one drug. In some instances, any one of the compositions disclosed herein further contains at least one anti-inflammatory drug. In some instances, the at least one anti-inflammatory drug is a nonsteroidal anti-inflammatory drug. In some instances, the nonsteroidal anti-inflammatory drug is acetylsalicylic acid, ibuprofen, ketoprofen, naproxen, one or more disease-modifying antirheumatic drug, salts thereof, or any combination thereof.

In some instances, the at least one anti-inflammatory drug is a disease-modifying antirheumatic drug (DMARD). In some instances, the at least one anti-inflammatory drug is a biological agent disease-modifying antirheumatic drug (biologic DMARD). In some instances, the biologic DMARD is adalimumab, a salt thereof, or any combination thereof. In some instances, the DMARD is methotrexate, a salt thereof, or any combination thereof.

In some aspects, any one of the compositions disclosed herein is formulated for oral administration. In some instances, the composition is a lozenge, a powder, a tablet, a gel-cap, a gelatin, a liquid solution, a syrup, an oil, and/or a dissolvable film. In some aspects, any one of the compositions disclosed herein is formulated for administration through injection. In some aspects, any one of the compositions disclosed herein is formulated for topical application and/or intranasal administration.

In some aspects, any one of the compositions disclosed herein is formulated to decrease inflammation. In some aspects, any one of the compositions disclosed herein is formulated to inhibit at least one proinflammatory cytokine. In some instances, the proinflammatory cytokine inhibited is TNF-α and/or IL-6. In some aspects, any one of the compositions disclosed herein is formulated to inhibit a prostaglandin. In some aspects, any one of the compositions disclosed herein is formulated to inhibit PGE-2. In some aspects, any one of the compositions disclosed herein is formulated to treat rheumatoid arthritis. In some aspects, any one of the compositions disclosed herein is formulated to prevent rheumatoid arthritis. In some aspects, any one of the compositions disclosed herein is formulated to prevent and/or treat polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis.

Methods of use for the compositions disclosed herein are also disclosed. In some aspects, a method is disclosed of treating a subject at risk for rheumatoid arthritis or having rheumatoid arthritis, the method includes administering any one of the compositions disclosed herein, wherein at least one symptom of rheumatoid arthritis is ameliorated in the subject and/or the onset of rheumatoid arthritis is delayed in comparison to the expected onset of rheumatoid arthritis if the patient had not been treated. In some instances, the subject is diagnosed as having rheumatoid arthritis.

In some aspects, a method is disclosed of treating a subject at risk for or having any one or more of the following diseases: polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis, the method includes administering any one of the compositions disclosed herein, wherein at least one symptom of the disease is ameliorated in the subject and/or the onset of the disease is delayed in comparison to the expected onset of the disease if the patient had not been treated. In some instances, the subject is diagnosed as having polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis.

In some instances, any one of the methods disclosed herein includes wherein the subject is administered a total amount of between 1 and 10,000 mg, between 10 and 5,000 mg, between 50 and 2,500 mg, or between 100 and 1,000 mg of the biomarker(s) during a 24 hour period. In some instances, any one of the methods disclosed herein includes wherein at least one of the biomarker(s) 1 through 22 is synthetically obtained. In some instances, any one of the methods disclosed herein includes wherein at least one of the biomarker(s) 1 through 22 is isolated from a plant. In some instances, any one of the methods disclosed herein includes wherein at least one of the biomarker(s) is isolated from Curcuma longa. In some instances, any one of the methods disclosed herein includes wherein the composition has an at least 95% batch-to-batch chemical consistency of relative abundance for the biomarkers.

In some aspects, any one of the methods disclosed herein includes wherein the composition further comprises at least one anti-inflammatory drug. In some instances, any one of the methods disclosed herein includes wherein the at least one anti-inflammatory drug is a nonsteroidal anti-inflammatory drug. In some instances, the nonsteroidal anti-inflammatory drug is acetylsalicylic acid, ibuprofen, ketoprofen, naproxen, one or more of disease-modifying antirheumatic drug, salts thereof, or any combination thereof. In some instances, the at least one anti-inflammatory drug is a disease-modifying antirheumatic drug (DMARD). In some instances, the at least one anti-inflammatory drug is a biological agent. In some instances, the at least one anti-inflammatory drug is a biological agent DMARD (biologic DMARD). In some instances, the at least one anti-inflammatory drug is adalimumab, a salt thereof, or any combination thereof. In some instances, the at least one anti-inflammatory drug is methotrexate, a salt thereof, or any combination thereof.

In some aspects, any one of the methods disclosed herein includes wherein the composition is administered orally. In some instances, the composition is administered as a lozenge, a powder, a tablet, a gel-cap, a gelatin, a liquid solution, a syrup, an oil, and/or a dissolvable film. In some aspects, any one of the methods disclosed herein includes wherein the composition is administered through injection. In some aspects, any one of the methods disclosed herein includes wherein the composition is administered topically and/or through intranasal administration.

In some aspects, any one of the methods disclosed herein includes wherein a proinflammatory cytokine is inhibited. In some aspects, any one of the methods disclosed herein includes wherein TNF-α and/or IL-6 is inhibited. In some aspects, any one of the methods disclosed herein includes wherein an prostaglandin is inhibited. In some aspects, any one of the methods disclosed herein includes wherein PGE-2 is inhibited.

In some aspects, a method is disclosed of reducing inflammation in a subject, the method includes administering any one of the compositions disclosed herein to a subject, wherein inflammation in the subject is reduced. In some aspects, a method is disclosed of preventing inflammation in a subject, the method includes administering any one of the compositions disclosed herein to a subject, wherein inflammation in the subject is prevented. In some aspects, a method is disclosed of inhibiting proinflammatory cytokine production and/or secretion in a subject, the method includes administering any one of the compositions disclosed herein to a subject, wherein the production and/or secretion of a proinflammatory cytokine is reduced. In some instances, the proinflammatory cytokine is TNF-α. In some instances, the proinflammatory cytokine is IL-6. In some aspects, a method is disclosed of inhibiting prostaglandin production and/or secretion in a subject, the method includes administering any one of the compositions disclosed herein to a subject, wherein the production and/or secretion of a prostaglandin is reduced. In some instances, the prostaglandin is PGE-2.

Methods of producing the compositions disclosed herein are also disclosed. In some aspects, a method is disclosed of producing any one of the compositions disclosed herein, wherein the method of producing produces a composition having an at least 90%, preferably at least 95% or at least 98% batch-to-batch chemical consistency of relative abundance for the biomarkers.

In some aspects of the invention, the composition may further comprise one or more nutraceutical and/or pharmaceutically acceptable carriers or diluents. These carriers/diluents can be natural products or non-naturally occurring. These carriers/diluents can be adjuvants, excipients, or vehicles such as preserving agents, fillers, disintegrating agents, wetting agents, emulsifiers, suspending agents, sweeteners, flavorings, fragrance, antibacterial agents, antifungal agents, lubricating agents, vitamins, polymers, siloxane containing compounds, essential oils, structuring agents, and dispensing agents. Each carrier is acceptable in the sense of being compatible with the other ingredients of the formulation and not injurious to the subject. In some aspects of the invention, the carrier can include at least one hydrophilic polymeric compound selected from the group consisting of a gum, a cellulose ether, an acrylic resin, a carbohydrate carrier, talc, lactose, mannitol, glucose, water, gelatin, a protein-derived compound, polyvinyl pyrrolidone, magnesium stearate, and any combination thereof. Non-limiting examples of diluents/carriers are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

The composition can be stored for one month, 6 months, 12 months, 18 months, or 24 months at room temperature. In some aspects of the invention, the composition is formulated as a powder, a tablet, a gel-cap, a bead, an edible tablet, a dissolvable film, a liquid capable of being dispersed through the air, a gelatin, a lotion, a transdermal patch, or a liquid solution for oral administration. In some aspects of the invention, the formulated composition can be comprised in a solid nanoparticle, a lipid-containing nanoparticle, a lipid-based carrier, a sealed conduit, a straw, sealed bag, or any combination thereof. In other aspects of the invention, the composition can be formulated for administration by injection.

Kits that include the compositions of the present invention are also contemplated. In certain embodiments, the composition is comprised in a container. The container can be a bottle, dispenser, package, or a straw. The container can dispense a predetermined amount of the composition. In certain aspects, the compositions are dispensed as a pill, a tablet, a capsule, a transdermal patch, an edible chew, a cream, a lotion, a gel, spray, mist, dollop, a powder, or a liquid. The container can include indicia on its surface. The indicia can be a word, an abbreviation, a picture, or a symbol.

It is contemplated that any embodiment discussed in this specification can be implemented with respect to any method or composition of the invention, and vice versa. Furthermore, compositions of the invention can be used to achieve methods of the invention.

Also contemplated is a product that includes the composition of the present invention. In non-limiting aspects, the product can be a nutraceutical product. The nutraceutical product can be those described in other sections of this specification or those known to a person of skill in the art. In other non-limiting aspects, the product can be a pharmaceutical product. The pharmaceutical and/or nutraceutical product can be those described in other sections of this specification or those known to a person of skill in the art. Non-limiting examples of products include a pill, a tablet, an edible chew, a capsule, a cream, a lotion, a gel, a spray, a mist, a dissolving film, a transdermal patch, or a liquid, etc.

Also disclosed are the following Embodiments 1 to 66 of the present invention. Embodiment 1 is a composition comprising any one of, or any combination of, or all of the following biomarkers: biomarker 11 having an accurate mass of 232.146 amu and having a relative abundance of at least 2.53%; biomarker 1 having an accurate mass of 146.113 amu and having a relative abundance of at least 0.20%; biomarker 2 having an accurate mass of 160.116 amu and having a relative abundance of at least 0.51%; biomarker 3 having an accurate mass of 176.128 amu and having a relative abundance of at least 0.35%; biomarker 4 having an accurate mass of 178.129 amu and having a relative abundance of at least 0.30%; biomarker 5 having an accurate mass of 180.106 amu and having a relative abundance of at least 0.10%; biomarker 6 having an accurate mass of 194.131 amu and having a relative abundance of at least 0.21%; biomarker 7 having an accurate mass of 198.146 amu and having a relative abundance of at least 2.86%; biomarker 8 having an accurate mass of 204.188 amu and having a relative abundance of at least 4.51%; biomarker 9 having an accurate mass of 218.167 amu and having a relative abundance of at least 88.89%; biomarker 10 having an accurate mass of 220.178 amu and having a relative abundance of at least 5.15%; biomarker 12 having an accurate mass of 234.166 amu and having a relative abundance of at least 8.04%; biomarker 13 having an accurate mass of 236.177 amu and having a relative abundance of at least 0.80%; biomarker 14 having an accurate mass of 238.191 amu and having a relative abundance of at least 0.13%; biomarker 15 having an accurate mass of 248.145 amu and having a relative abundance of at least 0.54%; biomarker 16 having an accurate mass of 268.189 amu and having a relative abundance of at least 0.18%; biomarker 17 having an accurate mass of 316.209 amu and having a relative abundance of at least 0.20%; biomarker 18 having an accurate mass of 326.234 amu and having a relative abundance of at least 0.28%; biomarker 19 having an accurate mass of 334.212 amu and having a relative abundance of at least 0.16%; biomarker 20 having an accurate mass of 350.230 amu and having a relative abundance of at least 0.21%; biomarker 21 having an accurate mass of 436.338 amu and having a relative abundance of at least 0.90%; wherein the biomarkers are found in Curcuma longa; and wherein the relative abundance is relative to 25 mg/ml salicylic acid spiked in 500 ng/ml of the composition. Embodiment 2 is the composition of Embodiment 1, wherein the biomarkers contained therein have a relative abundance of at most: biomarker 11 of 4.70%; biomarker 1 of 0.37%; biomarker 2 of 0.94%; biomarker 3 of 0.65%; biomarker 4 of 0.55%; biomarker 5 of 0.19%; biomarker 6 of 0.39%; biomarker 7 of 5.32%; biomarker 8 of 8.38%; biomarker 9 of 165.08%; biomarker 10 of 9.56%; biomarker 12 of 14.94%; biomarker 13 of 1.49%; biomarker 14 of 0.25%; biomarker 15 of 1.01%; biomarker 16 of 0.33%; biomarker 17 of 0.38%; biomarker 18 of 0.52%; biomarker 19 of 0.30%; biomarker 20 of 0.39%; biomarker 21 of 1.66%; wherein the relative abundance is relative to 25 mg/ml salicylic acid spiked in 500 ng/ml of the composition. Embodiment 3 is the composition of any of Embodiments 1 to 2, further comprising biomarker 22, having an accurate mass of 216.151 amu. Embodiment 4 is the composition of Embodiment 3, comprising at least 5.54 μg/m1 of biomarker 22. Embodiment 5 is the composition of any of Embodiments 3 to 4, wherein the composition comprises at most 10.29 μg/m1 of biomarker 22. Embodiment 6 is the composition of any of Embodiments 1 to 5, comprising at least 2, 3, 4, 5, 6, 7, 8, 9, 10, 11, 12, 13, 14, 15, 16, 17, 18, 19, 20, 21, or 22 of biomarkers 1 through 22. Embodiment 7 is the composition of any of Embodiments 1 to 6, wherein the mass of each biomarker is the mass as determined by a Direct Analysis in Real Time-TOF (DART-TOF) mass spectrometer. Embodiment 8 is the composition of any one of Embodiments 1 to 7, wherein at least one of the biomarker(s) are synthetically obtained. Embodiment 9 is the composition of any one of Embodiments 1 to 8, wherein at least one of the biomarker(s) are isolated from a plant. Embodiment 10 is the composition of Embodiment 9, wherein at least one of the biomarkers(s) are isolated from Curcuma longa. Embodiment 11 is the composition of any one of Embodiments 1 to 10, wherein the composition has an at least 90%, preferably at least 95%, or at least 98% batch-to-batch chemical consistency of relative abundance for the biomarkers. Embodiment 12 is the composition of any one of Embodiments 1 to 11, wherein the composition further comprises a preservative. Embodiment 13 is the composition of any one of Embodiments 1 to 12, wherein the composition further comprises at least one drug. Embodiment 14 is the composition of any of Embodiments 1 to 13, wherein the composition further comprises at least one anti-inflammatory drug. Embodiment 15 is the composition of Embodiment 14, wherein the at least one anti-inflammatory drug is a nonsteroidal anti-inflammatory drug. Embodiment 16 is the composition of Embodiment 15, wherein the nonsteroidal anti-inflammatory drug is acetylsalicylic acid, ibuprofen, ketoprofen, naproxen, one or more disease-modifying antirheumatic drug (DMARD), salts thereof, or any combination thereof. Embodiment 17 is the composition of Embodiment 14, wherein the at least one anti-inflammatory drug is methotrexate, a salt thereof, or any combination thereof. Embodiment 18 is the composition of Embodiment 14, wherein the at least one anti-inflammatory drug is a disease-modifying antirheumatic drug (DMARD). Embodiment 19 is the composition of Embodiment 18, wherein the DMARD is a biologic agent DMARD (biologic DMARD). Embodiment 20 is the composition of Embodiment 19, wherein the biologic DMARD is adalimumab, a salt thereof, or any combination thereof. Embodiment 21 is the composition of any one of Embodiments 1 to 20, wherein the composition is formulated for oral administration. Embodiment 22 is the composition of Embodiment 21, wherein the composition is a lozenge, a powder, a tablet, a gel-cap, a gelatin, a liquid solution, a syrup, an oil, and/or a dissolvable film. Embodiment 23 is the composition of any one of Embodiments 1 to 20, wherein the composition is formulated for administration through injection. Embodiment 24 is the composition of any one of Embodiments 1 to 20, wherein the composition is formulated for topical application and/or intranasal administration. Embodiment 25 is the composition of any of Embodiments 1 to 24, wherein the composition is formulated to decrease inflammation. Embodiment 26 is the composition of any of Embodiments 1 to 25, wherein the composition is formulated to inhibit at least one proinflammatory cytokine. Embodiment 27 is the composition of Embodiment 26, wherein the composition is formulated to inhibit TNF-α and/or IL-6. Embodiment 28 is the composition of any of Embodiments 1 to 27, wherein the composition is formulated to inhibit a prostaglandin. Embodiment 29 is the composition of any of Embodiments 1 to 28, wherein the composition is formulated to inhibit PGE-2. Embodiment 30 is the composition of any of Embodiments 1 to 29, wherein the composition is formulated to treat rheumatoid arthritis. Embodiment 31 is the composition of any of Embodiments 1 to 30, wherein the composition is formulated to prevent rheumatoid arthritis. Embodiment 32 is the composition of any of Embodiments 1 to 31, wherein the composition is formulated to treat polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. Embodiment 33 is the composition of any of Embodiments 1 to 32, wherein the composition is formulated to prevent polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. Embodiment 34 is a method of treating a subject at risk for or having any one or more of the following diseases: rheumatoid arthritis, polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis, the method comprising administering any one of the compositions of Embodiments 1 to 33 to the subject, wherein at least one symptom of the disease(s) is ameliorated in the subject and/or the onset of the disease(s) is delayed in comparison to the expected onset of the disease(s) if the patient had not been treated. Embodiment 35 is the method of Embodiment 34, wherein the subject is treated for rheumatoid arthritis or having rheumatoid arthritis, the method comprising administering any one of the compositions of Embodiments 1 to 33 to the subject, wherein at least one symptom of rheumatoid arthritis is ameliorated in the subject and/or the onset of rheumatoid arthritis is delayed in comparison to the expected onset of rheumatoid arthritis if the patient had not been treated. Embodiment 36 is the method of Embodiment 35, wherein the subject is diagnosed as having rheumatoid arthritis. Embodiment 37 is the method of Embodiment 34, wherein the subject is treated for or having any one or more of the following diseases: polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis, the method comprising administering any one of the compositions of Embodiments 1 to 33 to the subject, wherein at least one symptom of the disease(s) is ameliorated in the subject and/or the onset of the disease(s) is delayed in comparison to the expected onset of the disease(s) if the patient had not been treated. Embodiment 38 is the method of Embodiment 35, wherein the subject is diagnosed as having polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis

(Ps), non-infectious intermediate uveitis, non-infectious posterior uveitis, and/or non-infectious panuveitis uveitis. Embodiment 39 is the method of any one of Embodiments 34 to 38, wherein the subject is administered a total amount of between 1 and 10,000 mg, between 10 and 5,000 mg, between 50 and 2,500 mg, or between 100 and 1,000 mg of the biomarker(s) during a 24 hour period. Embodiment 40 is the method of any one of Embodiments 34 to 39, wherein at least one of the biomarker(s) 1 through 22 is synthetically obtained. Embodiment 41 is the method of any one of Embodiments 34 to 40, wherein at least one of the biomarker(s) 1 through 22 is isolated from a plant. Embodiment 42 is the method of Embodiment 41, wherein at least one of the biomarker(s) is isolated from Curcuma longa. Embodiment 43 is the method of any one of Embodiments 34 to 42, wherein the composition has an at least 95% batch-to-batch chemical consistency of relative abundance for the biomarkers. Embodiment 44 is the method of any of Embodiments 34 to 43, wherein the composition further comprises at least one anti-inflammatory drug. Embodiment 45 is the method of Embodiment 44, wherein the at least one anti-inflammatory drug is a nonsteroidal anti-inflammatory drug. Embodiment 46 is the method of Embodiment 45, wherein the nonsteroidal anti-inflammatory drug is acetylsalicylic acid, ibuprofen, ketoprofen, naproxen, one or more of disease-modifying antirheumatic drug (DMRD), salts thereof, or any combination thereof. Embodiment 47 is the method of Embodiment 44, wherein the at least one anti-inflammatory drug is methotrexate, a salt thereof, or any combination thereof. Embodiment 48 is the method of Embodiment 44, wherein the at least one anti-inflammatory drug is a disease-modifying antirheumatic drug (DMARD). Embodiment 49 is the method of Embodiment 48, wherein the DMARD is a biological agent DMARD (biologic DMARD). Embodiment 50 is the method of Embodiment 49, wherein the biologic DMARD is adalimumab, a salt thereof, or any combination thereof. Embodiment 51 is the method of any one of Embodiments 34 to 50, wherein the composition is administered orally. Embodiment 52 is the method of Embodiment 51, wherein the composition is administered as a lozenge, a powder, a tablet, a gel-cap, a gelatin, a liquid solution, a syrup, an oil, and/or a dissolvable film. Embodiment 53 is the method of any one of Embodiments 34 to 50, wherein the composition is administered through injection. Embodiment 54 is the method of any one of Embodiments 34 to 50, wherein the composition is administered topically and/or through intranasal administration. Embodiment 55 is the method of any of Embodiments 34 to 54, wherein a proinflammatory cytokine is inhibited. Embodiment 56 is the method of Embodiment 55, wherein TNF-α and/or IL-6 is inhibited. Embodiment 57 is the method of any of Embodiments 34 to 56, wherein an prostaglandin is inhibited.

Embodiment 58 is the method of any of Embodiments 34 to 57, wherein PGE-2 is inhibited. Embodiment 59 is a method of reducing inflammation in a subject, the method comprising administering the composition of any of Embodiments 1 to 33 to a subject, wherein inflammation in the subject is reduced. Embodiment 60 is a method of preventing inflammation in a subject, the method comprising administering the composition of any of Embodiments 1 to 33 to a subject, wherein inflammation in the subject is prevented. Embodiment 61 is a method of inhibiting proinflammatory cytokine production and/or secretion in a subject, the method comprising administering the composition of any of Embodiments 1 to 33 to a subject, wherein the production and/or secretion of a proinflammatory cytokine is reduced. Embodiment 62 is the method of Embodiment 61, wherein the proinflammatory cytokine is TNF-α. Embodiment 63 is the method of any of Embodiments 61 to 62, wherein the proinflammatory cytokine is IL-6. Embodiment 64 is a method of inhibiting prostaglandin production and/or secretion in a subject, the method comprising administering the composition of any of Embodiments 1 to 33 to a subject, wherein the production and/or secretion of a prostaglandin is reduced. Embodiment 65 is the method of Embodiment 64, wherein the prostaglandin is PGE-2. Embodiment 66 is a method of producing a composition of any of Embodiments 1 to 33, wherein the method of producing produces a composition having an at least 90%, preferably at least 95% or at least 98% batch-to-batch chemical consistency of relative abundance for the biomarkers.

“Therapeutic agent” encompasses the compounds specifically claimed herein. It also encompasses such compounds together with nutraceutical and/or pharmaceutically acceptable salts thereof. Useful salts are known to those skilled in the art and include salts with inorganic acids, organic acids, inorganic bases, or organic bases. Therapeutic agents useful in the present invention are those compounds that affect a desired, beneficial, and often pharmacological, effect upon administration to a human or an animal, whether alone or in combination with other nutraceutical and/or pharmaceutical excipients or inert ingredients.

The term “biomarker” refers to the compound defined as the biomarker, analogues thereof, derivatives thereof, salt forms thereof, or salt forms of any analogue or derivative thereof.

The term “accurate mass” refers to a measured mass of a molecule experimentally determined for an ion of known charge. The units for accurate mass include atomic mass units (amu) and milli unified atomic mass units (mmu). The term “molecular weight” refers to the average weight of the molecule with all of the different isotopic compositions present in a compound but weighted for their natural abundance.

The term “relative abundance” refers to the abundance of a compound of interest relative to the abundance of a reference compound. In particular aspects, relative abundance is the raw intensity of a mass spectrometry peak for the compound of interest over the raw intensity of a mass spectrometry peak for a reference compound. In one non-limiting instance, the mass spectrometry peaks can be obtained by the use of DART-TOF mass spectrometry. In another particular aspect, the reference compound is a compound that is spiked, or doped, into a sample containing the compound of interest. In yet another particular aspect, the reference compound is a compound that does not exist in the sample previous to its addition to the sample for determining relative abundance. In another particular aspect, the reference compound can be salicylic acid.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

“Patient,” “subject,” or “individual” refers to a mammal (e.g., human, primate, dog, cat, bovine, ovine, porcine, equine, mouse, rat, hamster, rabbit, or guinea pig). In particular aspects, the patient, subject, or individual is a human.

“Inhibiting” or “reducing” or any variation of these terms includes any measurable decrease or complete inhibition to achieve a desired result. The terms “promote” or “increase” or any variation of these terms includes any measurable increase or production of a protein or molecule to achieve a desired result.

“Effective” or any variation of this term means adequate to accomplish a desired, expected, or intended result. The result may include, but is not limited to any measurable change in an activity, production, a disease, a condition, or a symptom.

“Treating” or any variation of this term includes any measurable improvement in a disease, condition, or symptom that is being treated or is associated with the disease, condition, or symptom being treated.

“Preventing” or any variation of this term means to slow, stop, or reverse progression toward a result. The prevention may be any slowing of the progression toward the result.

“Analogue” and “analog,” when referring to a compound, refers to a modified compound wherein one or more atoms have been substituted by other atoms, or wherein one or more atoms have been deleted from the compound, or wherein one or more atoms have been added to the compound, or any combination of such modifications. Such addition, deletion or substitution of atoms can take place at any point, or multiple points, along the primary structure comprising the compound.

“Derivative,” in relation to a parent compound, refers to a chemically modified parent compound or an analogue thereof, wherein at least one substituent is not present in the parent compound or an analogue thereof. One such non-limiting example is a parent compound which has been covalently modified. Typical modifications are amides, carbohydrates, alkyl groups, acyl groups, esters, pegylations and the like.

A “therapeutically equivalent” compound is one that has essentially the same effect in the treatment of a disease or condition as one or more other compounds. A compound that is therapeutically equivalent may or may not be chemically equivalent, bioequivalent, or generically equivalent.

“Parenteral injection” refers to the administration of small molecule drugs via injection under or through one or more layers of skin or mucus membranes of an animal, such as a human.

“Bioavailability” refers to the extent to which the therapeutic agent is absorbed from the formulation.

“Pharmaceutically acceptable carrier” refers to a pharmaceutically acceptable solvent, suspending agent or vehicle for delivering a composition or drug compound of the present invention to a mammal such as an animal or human.

“Nutraceutically acceptable carrier” refers to a nutraceutical acceptable solvent, suspending agent or vehicle for delivering a compound of the present invention to an animal such as a mammal or human.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Nutraceutically acceptable” ingredient, excipient or component is one that is suitable for use with humans and/or animals without undue adverse side effects (such as toxicity, irritation and allergic response) commensurate with a reasonable benefit/risk ratio.

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the transitional phase “consisting essentially of,” in one non-limiting aspect, a basic and novel characteristic of the compositions and methods disclosed in this specification includes the compositions' abilities to reduce or prevent inflammation, rheumatoid arthritis, rheumatoid arthritis like symptoms, and/or related symptoms and/or causes such as, but not limited to inflammation.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Percentage inhibition by HSRx458 of TNF-α release from LPS challenged cells.

FIG. 2. Percentage inhibition by HSRx458 of IL-6 release from LPS challenged cells.

FIG. 3. Percentage inhibition by HSRx458 of PGE-2 release from LPS challenged cells.

### DETAILED DESCRIPTION

The inventors have surprisingly found that a combination of several compounds that can be found in turmeric can prevent and treat rheumatoid arthritis and inflammation. The inventors have also found that specific relative concentrations of the compounds act to enhance the ability of the combined compounds to prevent and treat rheumatoid arthritis and inflammation. In addition, the inventors have found that using compounds of the present invention with additional dugs enhance the ability of the combined compounds to prevent and treat rheumatoid arthritis and inflammation.

The compounds and compositions disclosed herein are capable of treating, ameliorating, and preventing the symptoms associated with rheumatoid arthritis and inflammation and side effects associated with the taking of drugs to treat rheumatoid arthritis and inflammation. Non-limiting examples of symptoms and/or causes of rheumatoid arthritis include inflammation of the synovium, bone erosion, joint deformity, pain, osteoporosis, carpal tunnel syndrome, hardened and blocked arteries, inflammation of the sac that encloses the heart, inflammation and scarring of lung tissue, fatigue, fever, weight loss, bumps of tissue under the skin of the arms, stiffness in the joints, and joints that are tender, warm, and swollen.

The inventors have also surprisingly found that the compounds and compositions disclosed herein can prevent and treat the following diseases: polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. Without wishing to be bound by theory, it is believed that at least some of the mechanisms of action of the compounds and compositions disclosed herein include those that address the underlying causes or symptoms of these diseases. Further, it is expected that using the compounds and compositions of the present invention with additional dugs will enhance the ability of the combined compounds to prevent and treat the diseases.

### A. Compounds of the Composition

In some aspects, the composition of the present invention can include one or more of the biomarkers found in Curcuma longa (turmeric) defined by accurate mass of 146.113 amu, 160.116 amu, 176.128 amu, 178.129 amu, 180.106 amu, 194.131 amu, 198.146 amu, 204.188 amu, 216.151 amu, 218.167 amu, 220.178 amu, 232.146 amu, 234.166 amu, 236.177 amu, 238.191 amu, 248.145 amu, 268.189 amu, 316.209 amu, 326.234 amu, 334.212 amu, 350.230 amu, and 436.338 amu, and combinations thereof. Without wishing to be bound by theory, it is believed that the biomarkers decrease inflammation.

In a particular embodiment, the biomarker or combination of biomarkers has a 90% batch-to-batch chemical consistency of relative abundance for the biomarkers. In another particular embodiment, the compound or combination of compounds has a 95% and/or 98% batch-to-batch chemical consistency of relative abundance for the biomarkers.

In some aspects of the invention, the compounds of the composition and derivatives and analogues can be made through known synthetic methods. In some aspects of the invention, the compounds of the composition and/or the composition can be synthetically obtained by producing the compound(s) and/or the compositions according to methods known to one of skill in the art in chemical synthesis. In some aspects, the compound(s) and/or the compositions are synthesized through organic chemistry methods.

In some aspects of the invention, the compounds of the composition and/or the composition can be isolated from extracts of an organism such as fruits, plants, animals, fungi, bacteria, and/or archaea. Non-limiting examples of plants include Curcuma longa. The compounds of the composition or the composition can be extracted from the organism using known extraction methods. Non-limiting examples of extractions capable of producing the compositions disclosed herein include contacting the extract with CO₂ at ranges of 35-70° C. and 50-350 Bar, or contacting the extract with H₂O or any combination of EtOH:H₂O, and separating the extract with any method utilizing polymer. A non-limiting example of a polymer used for polymer separation is ADS 5 polymer (Nankai University, China). The extract can include any one of or combination of compounds defined by accurate mass of 146.113 amu, 160.116 amu, 176.128 amu, 178.129 amu, 180.106 amu, 194.131 amu, 198.146 amu, 204.188 amu, 216.151 amu, 218.167 amu, 220.178 amu, 232.146 amu, 234.166 amu, 236.177 amu, 238.191 amu, 248.145 amu, 268.189 amu, 316.209 amu, 326.234 amu, 334.212 amu, 350.230 amu, and 436.338 amu that are found in Curcuma longa.

In some aspects of the invention, one or more of the compounds of the composition and derivatives and analogues thereof can be made through known synthetic methods known by one of skill in the art and one or more of the compounds of the composition and derivatives and analogues thereof may be isolated from other sources, such as, but not limited to, extracts of fruits and plants.

### B. Actives Defined by DART TOF/MS

The accurate mass and relative abundances described herein are based on experiments using particular instruments and particular settings and can change from instrument to instrument. There is variability in each measurement. Thus, the accurate mass and relative abundances are defined as being close to as understood by one of ordinary skill in the art. In one non-limiting embodiment the terms are defined to be within 20%, preferably 10%, preferably within 5%, more preferably within 1%, and most preferably within 0.5%. In one non-limiting embodiment, the accurate mass has an error of within +/−20 mmu, preferably 10 mmu, more preferably within 5 mmu, and most preferably within 1 mmu. In one non-limiting embodiment, the relative abundance has an error of +/−20%, preferably 10%, preferably within 5%, and more preferably within 1%, and most preferably within 0.5%.

In a non-limiting example, the compounds of the present invention can be identified using Direct Analysis in Real Time (DART) Time of Flight/Mass Spectrometry (TOF/MS). Specifically, a JEOL DART™ AccuTOF-mass spectrometer from Jeol USA of Peabody, Mass. (JMS-T100LC) can be used. The mass of compounds may be determined in a sample by directly introducing the sample to the ion stream by means of a Dip-IT sampler and a Dip-IT sampler holder (ionSense™). While no sample preparation is required for a simple analysis with the DART, a chemical doped/spiked solution can be used for quantitation relative to a known quantity. As a non-limiting example, the reference compound is not present in the sample until added to serve as a reference and can therefore be used to create a quantitative chemical profile of the bioactive molecules. The settings for the DART ion source can be the following:

Gas: He

Flow: 2.52 LPM @ 50 PSI

Temperature: 250 C

Needle Voltage: 3000V

Grid Electrode Voltage: 250V

Discharge Electrode Voltage: 400V

The settings for the JEOL AccuTOF MS can be the following:

Peaks Voltage: 1000V

Orifice 1 Temperature: 120 C

Detector Voltage: 2600V

Reflectron Voltage: 990.0V

Samples can be analyzed in six replicates by DART-TOF MS. These six replicates can be analyzed to create a single, averaged, filtered, and statistically significant DART fingerprint of the sample. This processed fingerprint can then be used to determine the presence of the bioactive markers by comparison of masses. Due to the initial discovery and identification of these bioactive markers, a simple mass comparison is sufficient to determine their presence in any extract or mixture of chemicals.

All mass spectrometers have a mass tolerance - a range of acceptable reported masses surrounding the predicted [M+H] or [M−H] value. For the AccuTOF, that mass tolerance is less than 20 millimass units (mmu) (predicted mass +/−10 mmu). Given the same sample and ion source, other TOF-MS may have a higher or lower mass tolerance.

In another non-limiting example, the compounds of the present invention can be determined by DART TOF/MS by using a JEOL DART™ AccuTOF-mass spectrometer from Jeol USA of Peabody, Mass. (JMS-T100LC) executed in the positive ion mode ([M+H]⁺) using the following settings for the DART ion source:

Gas: He

Flow: 3.98 L/min

Needle voltage: 3500 V

Temperature: 300 ° C.

Electrode 1 Voltage: 150 V

Electrode 2 Voltage: 250 V,

The settings for the JEOL AccuTOF MS can be the following:

Peaks Voltage: 1000V

Orifice 1 Voltage: 20 V

Ring Lens Voltage: 5 V

Orifice 2 Voltage: 5 V

Detector Voltage: 2550V

Calibrations can be performed internally with each sample using a 10% (weight/volume) solution of PEG 600 from Ultra Chemical of North Kingston, RI that provided mass markers throughout the required mass range of 100-1000 amu. Calibration tolerances can be held to 5 mmu. Samples can be introduced into the DART He plasma using the closed end of a borosilicate glass melting point capillary tube until a signal is achieved in the total-ion chromatogram (TIC). The next sample can then be introduced when the TIC returned baseline levels.

### C. Anti-Inflammatory Agents

It is contemplated that the compositions of the present invention can include anti-inflammatory agents. Anti-Inflammatory agents are compounds or compositions that are used to decrease the inflammatory response in a subject or decrease the effects of an inflammatory response. Non-limiting examples of anti-inflammatory agents include corticosteroids and nonsteroidal anti-inflammatory drugs. Non-limiting examples of nonsteroidal anti-inflammatory drugs include acetylsalicylic acid, ibuprofen, ketoprofen, naproxen, and DMARDs such as biological agent DMARDs, adalimumab, methotrexate, leflunomide, hydroxychloroquine, sulfasalazine, abatacept, anakinra, certolizumab, etanercept, golimumab, infliximab, rituximab, tocilizumab, and tofacitinib. Some anti-inflammatory drugs inhibit COX1 or COX2, or a pathway thereof. Some anti-inflammatory drugs inhibit 5LOX or the 5LOX pathway. Some anti-inflammatory agents reduce pro-inflammatory cytokines such as TNF-α and/or IL-6. In some embodiments, the compositions disclosed herein further include at least one additional anti-inflammatory agent, which may be, but is not limited to acetylsalicylic acid, ibuprofen, ketoprofen, naproxen, methotrexate, and biological agent DMARDs such as adalimumab, or any combination thereof.

### D. Amounts of Ingredients

It is contemplated that the compositions of the present invention can include any amount of the ingredients discussed in this specification. The compositions can also include any number of combinations of additional ingredients described throughout this specification (e.g., stabilizers, fillers, pharmaceutically and/or nutraceutical acceptable salts, and/or additional pharmaceutical and/or nutraceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition or relative abundance. A person of ordinary skill in the art would understand that the concentrations can vary depending on the addition, substitution, and/or subtraction of ingredients in a given composition.

### E. Additional Components

The compound of the present invention can be formulated into any suitable composition form for administration to a human or non-human animal patient.

The composition may consist of the claimed compounds alone or may include the compounds and any suitable additional component, such as one or more pharmaceutically and/or nutraceutical acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient.

2. Excipients

Excipients employed in the compositions of the present invention can be solids, semi-solids, liquids or combinations thereof. Preferably, the excipients are solids. Compositions of the invention containing excipients can be prepared by any known technique that comprises, for example, admixing an excipient with the claimed compounds. A pharmaceutical composition of the invention contains a desired amount of the claimed compounds per dose unit and, if intended for oral administration, can be in the form, for example, of a tablet, a caplet, a pill, a hard or soft capsule, a lozenge, a cachet, a dispensable powder, granules, a suspension, an elixir, an oil, a dispersion, or any other form reasonably adapted for such administration. If intended for intranasal administration, it can be in the form, for example, a dry powder, a nebulizer, or any other form reasonably adapted for such administration. If intended for parenteral administration, it can be in the form, for example, of a suspension, transdermal patch, or any other form reasonably adapted for such administration. If intended for rectal administration, it can be in the form, for example, of a suppository, or any other form reasonably adapted for such administration. Presently particular are oral dosage forms that are discrete dose units each containing a predetermined amount of the claimed compounds such as tablets or capsules.

3. Carriers/Diluents

Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC), calcium carbonate, glycine, clay, bentonite, block co-polymers, polyvinylpyrrolidone, and the like. Such carriers or diluents, if present, constitute in total about 5% to about 99.999%, about 10% to about 85%, and 20% to about 80%, of the total weight of the composition. The carrier, carriers, diluent, or diluents selected preferably exhibit suitable flow properties and, where tablets are desired, compressibility.

4. Disintegrant

Compositions of the invention optionally can include one or more pharmaceutically and/or nutraceutical acceptable disintegrants as excipients, particularly for tablet formulations. Suitable disintegrants include, but are not limited to, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. Disintegrants may be added at any suitable step during the preparation of the composition, particularly prior to granulation or during a lubrication step prior to compression. Such disintegrants, if present, constitute in total preferably about 0.2% to about 30%, preferably about 0.2% to about 10%, and more preferably about 0.2% to about 5%, of the total weight of the composition.

5. Binders

The compositions of the present invention can include binding agents or adhesives particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth, sucrose, gelatin, glucose, starches such as, but not limited to, pregelatinized starches, celluloses such as, but not limited to, methylcellulose and carmellose sodium, alginic acid and salts of alginic acid; magnesium aluminum silicate, PEG, guar gum, polysaccharide acids, bentonites, povidone, polymethacrylates, HPMC, hydroxypropylcellulose, and ethylcellulose. Such binding agents and/or adhesives, if present, constitute in total preferably about 0.5% to about 25%, preferably about 0.75% to about 15%, and more preferably about 1% to about 10%, of the total weight of the pharmaceutical composition. Many of the binding agents are polymers comprising amide, ester, ether, alcohol or ketone groups and, as such, can be included in pharmaceutical compositions of the present invention. Polyvinylpyrrolidones is an non-limiting example of a binder used for slow release tablets. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer may affect properties and performance.

6. Wetting Agents

Wetting agents can be used in the compositions of the present invention. Wetting agent can be selected to maintain the crystal in close association with water, a condition that may improve bioavailability of the composition. Such wetting agents can also be useful in solubilizing or increasing the solubility of crystals. Surfactants can be used as wetting agents. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, poloxamers (polyoxyethylene and polyoxypropylene block copolymers), polyoxyethylene fatty acid glycerides and oils, for example polyoxyethylene (8) caprylic/capric mono- and diglycerides, polyoxyethylene (35) castor oil and polyoxyethylene (40) hydrogenated castor oil, polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total preferably about 0.25% to about 15%, preferably about 0.4% to about 10%, and more preferably about 0.5% to about 5%, of the total weight of the pharmaceutical composition.

7. Lubricants

Lubricants can be included in the compositions of the present invention. Suitable lubricants include, but are not limited to, either individually or in combination, glyceryl behapate, stearic acid and salts thereof, including magnesium, calcium and sodium stearates; hydrogenated vegetable oils, colloidal silica, talc, waxes, boric acid, sodium benzoate, sodium acetate, sodium fumarate, sodium chloride, DL-leucine, PEG (e.g., Carbowax™ 4000 and Carbowax™ 6000 of the Dow Chemical Company), sodium oleate, sodium lauryl sulfate, and magnesium lauryl sulfate. Such lubricants, if present, constitute in total preferably about 0.1% to about 10%, preferably about 0.2% to about 8%, and more preferably about 0.25% to about 5%, of the total weight of the composition.

8. Other Agents

Surfactant, emulsifier, or effervescent agents can be used in the compositions. Emulsifying agents can be used to help solubilize the ingredients within a soft gelatin capsule. Non-limiting examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

### F. Vehicles

Various delivery systems are known in the art and can be used to administer a therapeutic agent or composition of the invention, e.g., encapsulation in liposomes, microparticles, microcapsules, receptor-mediated endocytosis and the like. Methods of administration include, but are not limited to, parenteral, intra-arterial, intramuscular, intravenous, intranasal, and oral routes. The compositions can be provided in the form of tablets, lozenges, granules, capsules, pills, ampoule, suppositories or aerosol form. The compositions can also be provided in the form of suspensions, solutions, and emulsions of the active ingredient in aqueous or non-aqueous diluents, syrups, oils, granulates or powders.

### G. Formulation and Administration

The composition may, for example, be a pharmaceutical composition (medicament), and over the counter composition (OTC), a nutraceutical, etc. Compositions according to the present invention include formulations suitable for nasal, oral, or parenteral routes. Non-limiting examples of specific routes include intradermal, subcutaneous, intramuscular, intravenous, local injection, rectal, intranasal inhalation, insufflation, topical (including transdermal, buccal and sublingual), vaginal, parenteral (including subcutaneous, intramuscular, intravenous and intradermal) and pulmonary administration. The formulations can conveniently be presented in unit dosage form and can be prepared by any methods well known in the art. Such methods include the step of bringing into association the active ingredient (or ingredients) with the carrier, which carrier constitutes one or more accessory ingredients. In general, the formulations are prepared by uniformly and intimately bringing into association the active ingredient with a suitable carrier, such as liquid carriers or finely divided solid carriers or both, and then if necessary shaping the product. Formulations of the subject invention suitable for oral administration can be presented as discrete units such as capsules, cachets or tablets, each containing a predetermined amount of the active ingredient, or as an oil-in-water liquid emulsion, water-in-oil liquid emulsion, or as a supplement within an aqueous solution, for example, a tea. The active ingredient can also be presented as bolus, electuary, or paste. Useful injectable preparations include sterile suspensions, solutions or emulsions of the compound compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives. Alternatively, the injectable formulation can be provided in powder form for reconstitution with a suitable vehicle, including but not limited to sterile pyrogen free water, buffer, dextrose solution, etc., before use. To this end, the compound compositions can be dried by any art-known technique, such as lyophilization, and reconstituted prior to use.

Formulations suitable for topical administration in the mouth include lozenges comprising the active ingredient in a flavored basis, usually sucrose and acacia or tragacanth, pastilles that include the active ingredient in an inert basis such as gelatin and glycerin, or sucrose and acacia, mouthwashes that include the active ingredient in a suitable liquid carrier, and chocolate comprising the active ingredients.

Formulations suitable for topical administration according to the subject invention can be formulated as an ointment, cream, suspension, lotion, oil, powder, solution, paste, gel, spray, aerosol or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream.

Formulations suitable for intranasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for intranasal administration, such as by the non-limiting examples of a nebulizer, include aqueous or oily solutions of the agent. Formulations preferably can include compounds that facilitate absorption of the active ingredients through the skin and into the bloodstream.

Formulations suitable for parenteral administration include: aqueous and non-aqueous isotonic sterile injection solutions which can contain antioxidants, buffers, bacteriostats and solutes which render the formulation isotonic with the blood of the intended recipient; aqueous and non-aqueous sterile suspensions which can include suspending agents and thickening agents; and liposomes or other microparticulate systems which are designed to target the compound to blood components or one or more organs. The formulations can be presented in unit-dose or multi-dose or multi-dose sealed containers, such as for example, ampoules and vials, and can be stored in a condition requiring only the addition of the sterile liquid carrier, for example, water for injections, immediately prior to use. Extemporaneous injection solutions and suspensions can be prepared from liquids, sterile powders, granules, and tablets of the kind previously described.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically and/or nutraceutical acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

For buccal administration, the compositions can take the form of the non-limiting examples of tablets or lozenges formulated in a conventional manner.

For rectal and vaginal routes of administration, the compound compositions can be formulated as solutions (for retention enemas) suppositories or ointments containing conventional suppository bases such as cocoa butter or other glycerides.

For nasal administration or administration by inhalation or insufflation, the compound compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

For prolonged delivery, the compound compositions can be formulated as a depot preparation for administration by implantation or intramuscular injection. The compound compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the compound compositions for percutaneous absorption, can be used. To this end, permeation enhancers can be used to facilitate transdermal penetration of the compound compositions. Suitable transdermal patches are described in for example, U.S. Pat. Nos. 5,407,713; 5,352,456; 5,332,213; 5,336,168; 5,290,561; 5,254,346; 5164,189; 5,163,899; 5,088,977; 5,087,240; 5,008,110; and 4,921,475.

Alternatively, other delivery systems can be employed. Liposomes and emulsions are well-known examples of delivery vehicles that can be used to deliver the compound compositions. Certain organic solvents such as dimethylsulfoxide (DMSO) can also be employed, although usually at the cost of greater toxicity.

It should be understood that in addition to the ingredients particularly mentioned above, the formulations useful in the present invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

In one embodiment, the pharmaceutical and/or nutraceutical compositions of the invention can be administered locally to the area in need of treatment; such local administration can be achieved, for example, by local infusion, by injection, or by means of a catheter. In another embodiment, a compound or composition of the invention is administered in a manner so as to achieve peak concentrations of the active compound at sites of the disease. Peak concentrations at disease sites can be achieved, for example, by intravenously injecting of the agent, optionally in saline, or orally administering, for example, a tablet, capsule or syrup containing the active ingredient.

H. Other Pharmaceutical and/or Nutraceutical Agents

Pharmaceutical, OTC, and/or nutraceutical formulations of the invention can be administered simultaneously or sequentially with other drugs or biologically active agents. Examples include, but are not limited to, antioxidants, free radical scavenging agents, analgesics, anesthetics, anorectals, antihistamines, anti-inflammatory agents including non-steroidal anti-inflammatory drugs, antibiotics, antifungals, antivirals, antimicrobials, anti-cancer actives, antineoplastics, biologically active proteins and peptides, enzymes, hemostatics, steroids including hormones and corticosteroids, etc.

### I. Therapeutic Methods And Dosage

Particular unit dosage formulations are those containing a daily dose or unit, daily subdose, or an appropriate fraction thereof, of an agent. Therapeutic amounts can be empirically determined and will vary with the pathology being treated, the subject being treated, and the efficacy and toxicity of the agent. Similarly, suitable dosage formulations and methods of administering the agents can be readily determined by those of ordinary skill in the art.

In some embodiments, a therapeutic method of the present invention can include treating a disease, condition, or disorder by administering to a subject having such disease or condition a stable formulation as described herein in an amount effective to treat the disease, condition, or disorder. In some embodiments, the subject is administered a stable formulation comprising the compounds claimed herein. The disease, condition, or disorder can be rheumatoid arthritis inflammation, and/or a disease with similar symptoms and related diseases, conditions, and disorders. For prophylactic administration, the composition can be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art. In some aspects of the invention, total dosage amounts of a compound composition will typically be in the range of from about 0.0001 or 0.001 or 0.01 mg/kg of patient/day to about 100 mg/kg patient/day, but may be higher or lower, depending upon, among other factors, the activity of the components, its bioavailability, the mode of administration and various factors discussed above. Dosage amount and interval can be adjusted individually to provide plasma levels of the compound(s) that are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation.

### J. Kits

In another aspect of the present invention, kits for treating a disease, condition or disorder as described herein. For instance, compositions of the present invention can be included in a kit. A kit can include a container. Containers can include a bottle, a metal tube, a laminate tube, a plastic tube, a dispenser, a straw, a pressurized container, a barrier container, a package, a compartment, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a predetermined amount of the composition. In other embodiments, the container can be squeezed (e.g., metal, laminate, or plastic tube) to dispense a desired amount of the composition. The composition can be dispensed as a spray, an aerosol, a liquid, a fluid, a semi-solid, or a solid. In a particular embodiment, the composition is dispensed as a tablet or lozenge. The containers can have spray, pump, or squeeze mechanisms. A kit can also include instructions for employing the kit components as well the use of any other compositions included in the container. Instructions can include an explanation of how to apply, use, and maintain the compositions. The compositions can, if desired, be presented in a pack or dispenser device, which can contain one or more unit dosage forms containing the compound compositions. The pack can, for example, comprise metal or plastic foil, such as a blister pack. The pack or dispenser device can be accompanied by instructions for administration.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Characterization of Compounds by Accurate Mass and Relative Abundance

The inventors have surprisingly found that a combination of several compounds can prevent and treat rheumatoid arthritis and inflammation. The inventors have also found that specific relative concentrations of the compounds act to enhance the ability of the combined compounds to prevent and treat these diseases. The compounds of the present invention include biomarker compounds defined by compounds found in Curcuma longa with an accurate mass of 146.113 amu, 160.116 amu, 176.128 amu, 178.129 amu, 180.106 amu, 194.131 amu, 198.146 amu, 204.188 amu, 216.151 amu, 218.167 amu, 220.178 amu, 232.146 amu, 234.166 amu, 236.177 amu, 238.191 amu, 248.145 amu, 268.189 amu, 316.209 amu, 326.234 amu, 334.212 amu, 350.230 amu, and 436.338 amu. These compounds may be produced synthetically or isolated from an organism such as, but not limited to, Curcuma longa. The compounds may be characterized by methods known by one of skill in the art.

Accurate mass and relative abundances described herein are based on experiments using particular instruments and particular settings and can change from instrument to instrument. There is variability in each measurement. Thus, the accurate mass and relative abundances are defined as being “close to” as understood by one of ordinary skill in the art.

Methods for Accurate Mass: The compounds were characterized and relative abundance was determined using Direct Analysis in Real Time (DART) ion source combined with Time of Flight/Mass Spectrometry (TOF-MS). Specifically, the DART TOF-MS was a JEOL DART™ AccuTOF-mass spectrometer from Jeol USA of Peabody, Mass. (JMS-T100LC). The mass of the compounds were determined in a Curcuma longa extract sample by directly introducing the sample to the ion stream by means of a Dip-IT sampler and a Dip-IT sampler holder (ionSense™). Accurate mass was determined by subtracting the mass of a proton (1.007825 amu) from the measured mass of the ions produced from the sample.

The settings for the DART ion source were the following:

Gas: He

Flow: 2.52 LPM @ 50 PSI

Temperature: 250 C

Needle Voltage: 3000V

Grid Electrode Voltage: 250V

Discharge Electrode Voltage: 400V

The settings for the JEOL AccuTOF MS were the following:

Peaks Voltage: 1000V

Orifice 1 Temperature: 120 C

Detector Voltage: 2600V

Reflectron Voltage: 990.0V

Extract samples were analyzed in six replicates by DART-TOF MS. These six replicates were analyzed to create a single, averaged, filtered, and statistically significant DART fingerprint of the extract. This processed fingerprint was then used to determine the presence of the bioactive markers by comparison of masses. Due to the initial discovery and identification of these bioactive markers, a simple mass comparison was sufficient to determine their presence in any extract or mixture of chemicals. For the AccuTOF, that mass tolerance is less than 20 millimass units (mmu) (predicted mass +/−10 mmu). Given the same extract and ion source, other TOF mass spectrometers may have a higher or lower mass tolerance.

Methods for Relative Abundance: While no sample preparation is required for a simple analysis with the DART, a salicylic acid doped/spiked solution was used for determining relative abundance of test compositions through quantitation relative to a known quantity. Standards that are well known and that exist naturally in turmeric, such as curcumin, would vary given any number of influences—growing conditions, harvest time, plant health, etc. For purposes of quantifying the biomarkers, the natural variations of curcumin (or other naturally occurring standards) make it unacceptable to use as a basis for an absolute quantification of the biomarkers. In order to remove that inconsistency, a compound that is not native to turmeric (in this case, salicylic acid) was used as the basis for a quantitative chemical profile of the bioactive molecules.

For determining relative abundance of samples with unknown concentrations of the biomarkers disclosed herein, 550 nl (500 ng) of a composition of biomarkers in the form of an anhydrous oil was diluted into 1 ml ethanol as a solvent (550 nl/m1 or 500 ng/ml) and doped/spiked with 25 mg/ml salicylic acid. Samples were then analyzed by the DART-TOF method used above.

Table 1 discloses the relative abundance of the biomarkers disclosed herein found in non-limiting, particular embodiments of compositions comprising all 22 biomarkers.

TABLE 1
Relative Abundance of the biomarkers in particular
active compositions determined using 500 ng/ml of
the composition and spiked with 25 mg/mL salicylic acid.

| | Accurate | Minimum Relative | Maximum Relative |
|---|---|---|---|
| | Mass | Abundance | Abundance |
| | (amu) | (−30%) | (+30%) |
| Biomarker 1 | 146.113 | 0.20% | 0.37% |
| Biomarker 2 | 160.116 | 0.51% | 0.94% |
| Biomarker 3 | 176.128 | 0.35% | 0.65% |
| Biomarker 4 | 178.129 | 0.30% | 0.55% |
| Biomarker 5 | 180.106 | 0.10% | 0.19% |
| Biomarker 6 | 194.131 | 0.21% | 0.39% |
| Biomarker 7 | 198.146 | 2.86% | 5.32% |
| Biomarker 8 | 204.188 | 4.51% | 8.38% |
| Biomarker 9 | 218.167 | 88.89% | 165.08% |
| Biomarker 10 | 220.178 | 5.15% | 9.56% |
| Biomarker 11 | 232.146 | 2.53% | 4.70% |
| Biomarker 12 | 234.166 | 8.04% | 14.94% |
| Biomarker 13 | 236.177 | 0.80% | 1.49% |
| Biomarker 14 | 238.191 | 0.13% | 0.25% |
| Biomarker 15 | 248.145 | 0.54% | 1.01% |
| Biomarker 16 | 268.189 | 0.18% | 0.33% |
| Biomarker 17 | 316.209 | 0.20% | 0.38% |
| Biomarker 18 | 326.234 | 0.28% | 0.52% |
| Biomarker 19 | 334.212 | 0.16% | 0.30% |
| Biomarker 20 | 350.230 | 0.21% | 0.39% |
| Biomarker 21 | 436.338 | 0.90% | 1.66% |
| Biomarker 22 | 216.151 | 5.54 μg/mL | 10.29 μg/mL |

### Example 2

### HSRx458 Formulation

A particular embodiment, HSRx458, of the disclosed composition was prepared as an oil that comprises a dose-reliable, turmeric extract comprising biomarkers 1 through 22 at the following relative abundances compared to 25 mg/mL salicylic acid spiked into 500 ng/ml of the composition or concentrations: Biomarker 1-0.28%; Biomarker 2-0.73%; Biomarker 3- 0.50%; Biomarker 4-0.43%; Biomarker 5-0.14%; Biomarker 6-0.30%; Biomarker 7- 4.09%; Biomarker 8-6.44%; Biomarker 9-126.98%; Biomarker 10-7.35%; Biomarker 11-3.62%; Biomarker 12-11.49%; Biomarker 13-1.15%; Biomarker 14-0.19%; Biomarker 15-0.78%; Biomarker 16-0.26%; Biomarker 17-0.29%; Biomarker 18-0.40%; Biomarker 19-0.23%; Biomarker 20-0.30%; Biomarker 21-1.28%; Biomarker 22-7.92 μg/mL. This embodiment has in vitro and in vivo activity against the causes and symptoms of rheumatoid arthritis and inflammation. This embodiment was tested as a DMSO dissolved liquid for the in vitro cellular studies or will be tested in capsule form in human trials.

Non-limiting examples of methods to produce HSRx458 include the following general methods. Turmeric (Curcuma longa) was ground and extracted by contacting the extract with CO₂ at ranges of 35-70° C. and 50-350 Bar, or contacting the extract with H₂Oor any combination of EtOH:H₂O, and separating the extract with any method utilizing polymer. A non-limiting example of a polymer used for polymer separation is ADS 5 polymer (Nankai University, China).

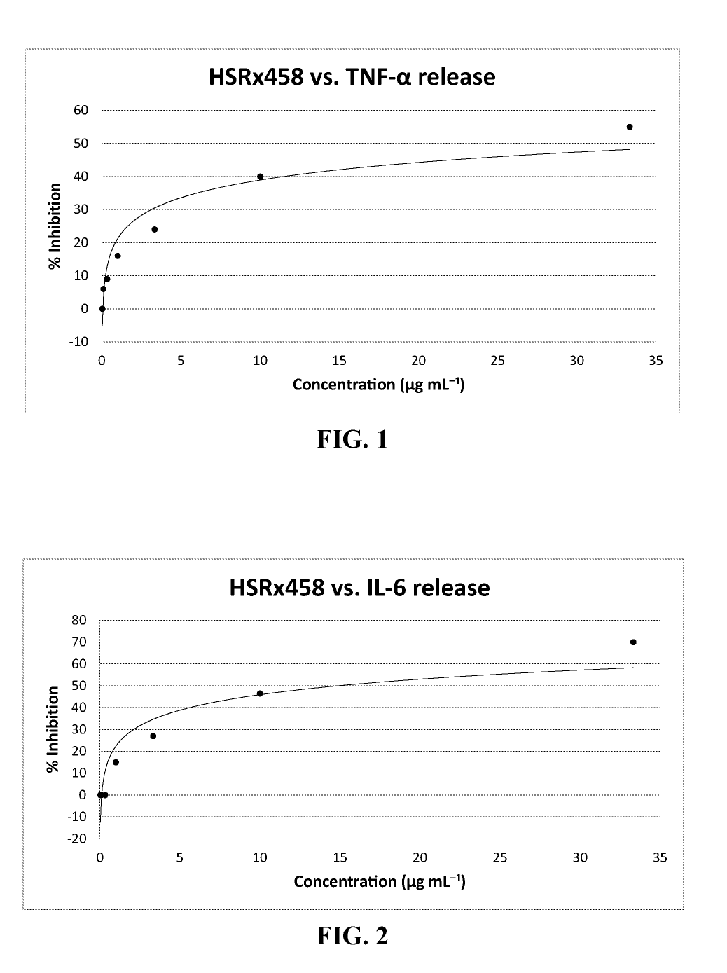

The composition disclosed herein inhibited TNF-α, IL-6, and PGE-2 production in lipopolysaccharide challenged cells as disclosed below and summarized in Table 2. The Curve fit was based on the curves represented in FIGS. 1, 2, and 3.

TABLE 2
HSRx458 IC₅₀ for TNF-α, IL-6, and PGE-2
production in lipopolysaccharide challenged cells.

| | IC₅₀ (μg/ml) | N | R² | Curve fit |
|---|---|---|---|---|
| TNF-α | 25.6 | 14 | 0.93 | y = 7.6955ln(x) + 21.197 |
| IL-6 | 14.9 | 14 | 0.88 | y = 10.2451ln(x) + 22.334 |
| PGE-2 | 8.4 | 12 | 0.81 | y = 8.6001ln(x) + 26.788 |

These results, as demonstrated below, indicate that the compositions and methods disclosed herein are effective for treating and/or preventing inflammation and diseases that are caused by or have inflammation as a symptom, such as, but not limited to, rheumatoid arthritis, polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis. Without wishing to be bound by theory, the compounds and compositions disclosed herein have been shown to inhibit TNF-α, IL-6, and PGE-2 (see Table 2). Similarly, adalimumab, a treatment for rheumatoid arthritis is known to also inhibit TNF-α. Thus, it is expected that the compounds and compositions disclosed herein will also be effective for treating the same diseases and conditions that adalimumab is effective for, including rheumatoid arthritis, polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis.

### Example 3

### Iinhibition of TNF-α and IL-6 Release

TNF-α and IL-6 release activities in response to a lipopolysaccharide (LPS) challenge were assessed in the presence or absence of HSRx458 dissolved in DMSO. Activity was calculated as the percentage of release relative to the negative controls. IC₅₀ values were calculated and found to be 25.6 μg/ml for TNF-α inhibition and 14.9 μg/ml for IL-6 inhibition.

Briefly, TNF-α and IL-6 inhibition assays were conducted by Sanofi, Bridgewater, N.J. Splenocyte suspensions were prepared from Balb/c mice. Splenocytes (7×10⁵/well) were added in 96 well plate with 10% FBS in RPMI1640 medium. Splenocytes were pre-treated with or without HSRx458 for 1 hour, and then LPS (1 μg/m1) was added followed by incubation at 37° C. with 5% CO2 for 18 hours. Cytotoxicity was measured using the Promega kit (Part Number G3582) and supernatants were collected in order to measure TNF-α and IL-6 by ELISA (R&D kits, DY410 an DY406). Percent inhibition of TNF-α and IL-6 release relative to control wells was calculated at each concentration of HSRx458 tested.

The percent inhibition at each concentration of HSRx458 tested were plotted to determine the IC₅₀ for inhibition of TNF-α and IL-6 (FIG. 1 and FIG. 2, respectively). The HSRx458 50% inhibition values were determined using data generated by Sanofi for each endpoint. In all cases logarithmic ‘best fit’ analyses were used when the r² values for the line fit was ≥0.85. In cases where the r² values for the line fit was <0.85 and if the extract reached an IC₅₀ value, an IC₅₀ value was estimated by determining the intersection of line-of-best-fit (linear data) and the 50% inhibition line (y-axis). It was determined that the IC₅₀ values were 25.6 μg/ml for TNF-α inhibition and 14.9 μg/ml for IL-6 inhibition.

### Example 4

### Inhibition of PGE-2 Release

PGE-2 release activity in response to a lipopolysaccharide (LPS) challenge were assessed in the presence or absence of HSRx458 dissolved in DMSO. Activity was calculated as the percentage of release relative to the negative controls. The IC₅₀ value was calculated and found to be 8.4 μg/ml for PGE-2 inhibition.

Briefly, PGE-2 inhibition assays were conducted by Sanofi-Aventis, Bridgewater, N.J. Assays were conducted independently on two separate days to quantify the PGE-2 secretion from LPS (Sigma-Aldrich, St. Louis, Mo.) stimulated RAW 264.7 cells. RAW 264.7 cells (ATCC # TIB-71) were grown and maintained in DMEM medium (Invitrogen, Irvine, Calif.) containing 10% fetal bovine serum (Invitrogen, Irvine, Calif.). One day prior to the experiment, the cells were split and fed fresh medium. On the day of the study, the RAW 264.7 cells were seeded at 2.5×10⁴ cells/well in a 96 well flat bottom plate and incubated at 37° C. in a 5% CO₂ incubator for 1 hour. 10 μL of HSRx458 or 50× diluted Celebrex (Toronto Research Chemicals Inc, Canada) were added to the designated wells and further incubated for 30 minutes in the incubator. The RAW 264.7 cells were stimulated with 100 μL of medium containing 2 μg mL⁻¹ of LPS in all wells except wells marked “None”. These control wells received 100 μL of medium alone. The cells were cultured for 21 hours. 180 μL of the supernatant was harvested from each well and PGE-2 levels in the supernatant medium were quantified using an EIA PGE-2 Kit (Assay Designs, Ann Arbor, Mich.) following the manufacturer's instructions. Cell viability was measured by adding 100 μL of the medium: MTT mix (80:20) (Promega, Madison, Wis.) following manufacturer's instructions (20 μL of Cell Titer 96 Aqueous One Solution mixed with 80 μL of the medium). Plate absorbance was read at 490 nm. Percent inhibition of PGE-2 release relative to control wells was calculated at each concentration of HSRx458 tested.

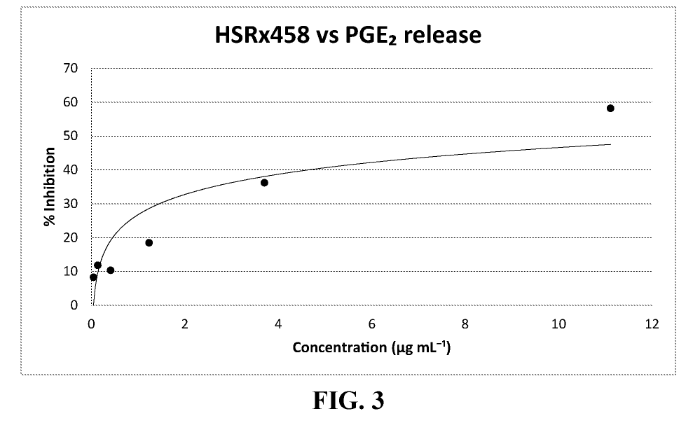

The percent inhibition at each concentration of HSRx458 tested were plotted to determine the IC₅₀ for inhibition of PGE-2 (FIG. 3). The HSRx458 50% inhibition value was determined using data generated by Sanofi for each endpoint. In all cases logarithmic ‘best fit’ analyses were used when the r² values for the line fit was ≥0.85. In cases where the r² values for the line fit was <0.85 and if the extract reached an IC₅₀ value, an IC₅₀ value was estimated by determining the intersection of line-of-best-fit (linear data) and the 50% inhibition line (y-axis). It was determined that the IC₅₀ is 8.4 μg/ml for PGE-2 inhibition.

### Example 5

### Anti-Inflammatory Capacity

This example concerns a planned double blind clinical trial using HSRx458 to determine the safety and tolerability of HSRx458 and its effects on decreasing inflammatory cytokines in humans.

Lipopolysaccharide (LPS) challenge is a model system to study systemic inflammation without underlying disease. Injection of healthy volunteers with low doses of LPS (a.k.a., endotoxin) increases inflammatory markers, such as TNF-α and IL-6, known to be associated with a number of disease conditions, including rheumatoid arthritis.

The primary aim of the study is to identify healthy individuals and demonstrate that the HSRx458 product successfully reduces mediators of inflammation associated with rheumatoid arthritis (TNF-α, IL-6), versus a placebo. Specifically, the study is designed to: 1) measure the reduction in TNF-α and IL-6 measured at 1, 1.5, 2, 2.5, 3, 4, and 6 hours, comparing volunteers using HSRx458 versus a placebo; 2) measure the change in reported clinical symptoms, such as headache, chills, etc. rated on a scale of 0-3; 3) measure the change in clinical signs, including temperature, heart rate, and blood pressure; and 4) determine adverse side effects throughout the course of the study.

Methodology: Sixteen (16) healthy human volunteers between the age of 18 and 35 years of age will be picked for this study. The volunteers will not have taken any immunosuppressive drugs in the last 12 months, not taken any insulin or warfarin in the last 12 weeks, not taken any diabetic medications or antibiotic or anti-viral medications in the last 4 weeks, and not taken any NSAIDs, antihistamines, or omega 3-fatty acid dietary supplements for at least 1 week before the enrollment in the this study. The volunteers will agree not to take any NSAIDs/antihistamines or other pain/inflammation medication during the course of the study, not to initiate any new exercise or diet program, and not to change their current diet and exercise program during the course of the study.

The total study duration will be 6 hours and will include the following components.

Visit 1, Baseline:
1) Volunteers will be randomized 1:1 into each of two study arms to receive either an oral dose of 175 mg of HSRx458 or oral placebo containing maltodextrin 15 minutes prior to intravenous (i.v.) administration of 2 ng/kg E. coli LPS, a known mediator of inflammation and inflammatory cytokines.
2) Vital Signs: Vital signs, including blood pressure, pulse rate, respiratory rate and oral body temperature will be measured after the volunteer has rested in a seated position for at least 5 minutes. Height and weight measurements will be collected at Visit 1.
3) Limited Physical Exam: Staff will perform a follow-up physical examination that will at a minimum include the following: assessment of general appearance, HEENT (head, eyes, ears, nose and throat), heart, lungs, musculoskeletal system, and lymph nodes.
4) Clinical symptoms: Volunteers will be evaluated on a scale of 0-3 for each of the following clinical symptoms: headache, chills, myalgia, nausea, vomiting, abdominal pain, and backache.
5) Clinical Laboratory Testing: Blood samples will be collected immediately prior to ingestion of study medication or placebo and then again immediately before LPS administration and sent to a certified and registered laboratory for processing for the detection and measurement of TNF-α and IL-6 inflammation markers.

Visit 1, Hour 1 through Final Visit, Hour 6:
1) Clinical signs and symptoms: Volunteers will be evaluated on a scale of 0-3 for each of the following clinical symptoms: headache, chills, myalgia, nausea, vomiting, abdominal pain, and backache. Staff will take vital signs, including blood pressure, pulse rate, respiratory rate and oral body temperature.
2) Clinical Laboratory Testing: Blood samples will be collected at 1, 1.5, 2, 2.5, 3, 4, and 6 hours post-injection of LPS and sent to a certified and registered laboratory for processing for the detection and measurement of TNF-α and IL-6 inflammation markers.
3) Review of Adverse Events: The volunteer will be queried for any adverse events.

Outcomes Measured: The following outcomes will be measured in this clinical trial. TNF-α concentration, IL-6 concentration, temperature, heart rate, and blood pressure at each interval will be measured by staff. Staff will record study volunteer reports of headache, chills, myalgia, nausea, vomiting, abdominal pain, and backache at each interval on a scale of 0 to 3 (0=absent, 1=mild, 2=moderate, 3=severe). Staff will record any evidence of adverse events.

Statistical Analysis: Statistical analysis of the results will be performed.

### Example 6

### Synergy

The predicted method of action of the biomarkers disclosed herein is believed to enable the biomarkers to act synergistically with other compounds that act through a separate mechanism to treat or prevent rheumatoid arthritis and/or inflammation. To further confirm such synergism and determine synergism with other compounds/compositions, one or more of the biomarkers disclosed herein can be tested in combination with one or more of the other biomarkers disclosed herein, and/or one or more drugs and/treatments. Combination studies can show competitive, additive, or synergistic interactions for treatment and/or prevention of rheumatoid arthritis and/or inflammation and/or the symptoms thereof in cell culture, animal studies, human studies, etc. Non-limiting examples of studies can include those described above and herein as well as those known to one of skill in the art. As a non-limiting example, the combination of HSRx458 and NSAIDs may be tested.

A non-limiting example of a combination assay that can be performed to determine the competitive, additive, or synergistic interactions of a combination can utilize an interaction matrix commonly used to look at drug interactions and synergy. In one instance, the interaction matrix is used in a prevention or treatment study of inflammation in cell culture. Briefly, the experiment can have 25 samples: 4 with a first test compound/composition (such as HSRx458) alone, 4 with a second test compound/composition (such as adalimumab) alone, 1 with no chemistries, and the remaining 16 can be combinations of the first and second test compounds/compositions. 1:4 dilutions of the first test compound/composition from a starting concentration (such as 1 mg/ml for HSRx458) and 1:4 dilutions of the second test compound/composition from a starting concentration (such as 1.0 μg/ml for adalimumab) can be tested. The ability to decrease inflammation markers, etc. can occur in the constant presence of the inhibitory compounds. In this way, the experiment simulates a patient while on prophylactic treatment and tests prevention of disease onset by the first test compound/composition alone, the second test compound/composition alone, and the combination of the two at a range of concentrations. The data can be analyzed with the methodology of Berenbaum to determine competitive, additive, or synergistic interactions. (Berenbaum 1977).

All of the compositions and/or methods disclosed and claimed herein can be made and executed without undue experimentation in light of the present disclosure. While the compositions and methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Diseases and Conditions, Rheumatoid arthritis: Mayo Clinic. Oct. 29,2014. [cited 2015 Oct. 16]. http://www.mayoclinic.org/diseases-conditions/rheumatoid-arthritis/basics/definition/con-20014868
Andreasson K. (2010) Emerging roles of PGE2 receptors in models of neurological disease. Prostaglandins Other Lipid Mediat 91(3-4):104-112.
Berenbaum M C. (1977) Synergy, additivism and antagonism in immunosuppression. A critical review. Clin Exp. Immunol. 28(1):1-18.
Choi, et al. (2014) Sulforaphane inhibits IL-1β-induced proliferation of rheumatoid arthritis synovial fibroblasts and the production of MMPs, COX-2, and PGE2. Inflammation 37(5): 1496-1503.
Cody R B, Laramee J A, and Durst H D. (2005) Versatile new ion source for the analysis of materials in open air under ambient conditions. Analytical Chemistry 77:2297-2302.
Gottenberg J E, et al. (2012) Serum IL-6 and IL-21 are associated with markers of B cell activation and structural progression in early rheumatoid arthritis: results from the ESPOIR cohort. Annals of the Rheumatic Diseases 71:1243-1248.
Hennigan S, et al. (2008) Interleukin-6 inhibitors in the treatment of rheumatoid arthritis. Ther Clin Risk Manag 4:767-775.
Huwiler et al. (2009) Lipids as targets for novel anti-inflammatory therapies. Pharmacol Ther 124:96-112.
Kremer et al. (2011) Tocilizumab inhibits structural joint damage in rheumatoid arthritis patients with inadequate responses to methotrexate: Results from the double-blind treatment phase of a randomized placebo-controlled trial of tocilizumab safety and prevention of structural joint damage at one year. Arthritis & Rheumatism 63:609-621.
Nishimoto et al. (2006) Interleukin 6: from bench to bedside. Nat Clin Pract Rheumatol 2:619-626.
Nishimoto et al. (2014) Retreatment efficacy and safety of tocilizumab in patients with rheumatoid arthritis in recurrence (RESTORE) study. Modern Rheumatology 24:26-32.

Park et al. (2006) Prostaglandin E2 synthesis and secretion: The role of PGE2 synthases. Clin Immunol 119:229-240.
Qin et al. (2014) Cinnamon polyphenols increase oxygen-glucose deprivation of decreased PGE2 production by upregulation of SIRT1 and alleviation of the anti-inflammatory effects (830.11). The FASEB Journal 28.
Wei Zuo et al. (2011) Adiponectin receptor 1 mediates the difference in adiponectin-induced prostaglandin E2 production in rheumatoid arthritis and osteoarthritis synovial fibroblasts. Chinese Medical Jounral (124):6.

1. A composition comprising the following biomarkers:
biomarker 11 having an accurate mass of 232.146 amu and having a relative abundance of at least 2.53%;
biomarker 1 having an accurate mass of 146.113 amu and having a relative abundance of at least 0.20%;
biomarker 2 having an accurate mass of 160.116 amu and having a relative abundance of at least 0.51%;
biomarker 3 having an accurate mass of 176.128 amu and having a relative abundance of at least 0.35%;
biomarker 4 having an accurate mass of 178.129 amu and having a relative abundance of at least 0.30%;
biomarker 5 having an accurate mass of 180.106 amu and having a relative abundance of at least 0.10%;
biomarker 6 having an accurate mass of 194.131 amu and having a relative abundance of at least 0.21%;
biomarker 7 having an accurate mass of 198.146 amu and having a relative abundance of at least 2.86%;
biomarker 8 having an accurate mass of 204.188 amu and having a relative abundance of at least 4.51%;
biomarker 9 having an accurate mass of 218.167 amu and having a relative abundance of at least 88.89%;
biomarker 10 having an accurate mass of 220.178 amu and having a relative abundance of at least 5.15%;
biomarker 12 having an accurate mass of 234.166 amu and having a relative abundance of at least 8.04%;
biomarker 13 having an accurate mass of 236.177 amu and having a relative abundance of at least 0.80%;
biomarker 14 having an accurate mass of 238.191 amu and having a relative abundance of at least 0.13%;
biomarker 15 having an accurate mass of 248.145 amu and having a relative abundance of at least 0.54%;
biomarker 16 having an accurate mass of 268.189 amu and having a relative abundance of at least 0.18%;
biomarker 17 having an accurate mass of 316.209 amu and having a relative abundance of at least 0.20%;
biomarker 18 having an accurate mass of 326.234 amu and having a relative abundance of at least 0.28%;
biomarker 19 having an accurate mass of 334.212 amu and having a relative abundance of at least 0.16%;
biomarker 20 having an accurate mass of 350.230 amu and having a relative abundance of at least 0.21%; and
biomarker 21 having an accurate mass of 436.338 amu and having a relative abundance of at least 0.90%;
wherein the biomarkers are found in Curcuma longa; and
wherein the relative abundance is relative to 25 mg/ml salicylic acid spiked in 500 ng/ml of the composition.

2. The composition of claim 1, wherein the biomarkers contained therein have a relative abundance of at most:
biomarker 11 of 4.70%;
biomarker 1 of 0.37%;
biomarker 2 of 0.94%;
biomarker 3 of 0.65%;
biomarker 4 of 0.55%;
biomarker 5 of 0.19%;
biomarker 6 of 0.39%;
biomarker 7 of 5.32%;
biomarker 8 of 8.38%;
biomarker 9 of 165.08%;
biomarker 10 of 9.56%;
biomarker 12 of 14.94%;
biomarker 13 of 1.49%;
biomarker 14 of 0.25%;
biomarker 15 of 1.01%;
biomarker 16 of 0.33%;
biomarker 17 of 0.38%;
biomarker 18 of 0.52%;
biomarker 19 of 0.30%;
biomarker 20 of 0.39%; and
biomarker 21 of 1.66%;
wherein the relative abundance is relative to 25 mg/ml salicylic acid spiked in 500 ng/ml of the composition.

3. The composition of any of claims 1 to 2, further comprising biomarker 22, having an accurate mass of 216.151 amu.

4. The composition of claim 3, comprising at least 5.54 μg/ml of biomarker 22.

5. The composition of any of claims 3 to 4, wherein the composition comprises at most 10.29 μg/ml of biomarker 22.

6. The composition of any of claims 1 to 5, wherein the mass of each biomarker is the mass as determined by a Direct Analysis in Real Time-TOF (DART-TOF) mass spectrometer.

7. The composition of any one of claims 1 to 6, wherein at least one of the biomarker(s) are synthetically obtained.

8. The composition of any one of claims 1 to 7, wherein at least one of the biomarker(s) are isolated from a plant.

9. The composition of claim 8, wherein at least one of the biomarkers(s) are isolated from Curcuma longa.

10. The composition of any one of claims 1 to 9, wherein the composition has an at least 90%, preferably at least 95%, or at least 98% batch-to-batch chemical consistency of relative abundance for the biomarkers.

11. The composition of any one of claims 1 to 10, wherein the composition further comprises a preservative.

12. The composition of any one of claims 1 to 11, wherein the composition further comprises at least one drug.

13. The composition of any of claims 1 to 12, wherein the composition further comprises at least one anti-inflammatory drug.

14. The composition of claim 13, wherein the at least one anti-inflammatory drug is a nonsteroidal anti-inflammatory drug.

15. The composition of claim 14, wherein the nonsteroidal anti-inflammatory drug is acetylsalicylic acid, ibuprofen, ketoprofen, naproxen, one or more disease-modifying antirheumatic drug (DMARD), salts thereof, or any combination thereof.

16. The composition of claim 13, wherein the at least one anti-inflammatory drug is methotrexate, a salt thereof, or any combination thereof.

17. The composition of claim 13, wherein the at least one anti-inflammatory drug is a disease-modifying antirheumatic drug (DMARD).

18. The composition of claim 17, wherein the DMARD is a biologic agent DMARD (biologic DMARD).

19. The composition of claim 18, wherein the biologic DMARD is adalimumab, a salt thereof, or any combination thereof.

20. The composition of any one of claims 1 to 19, wherein the composition is formulated for oral administration.

21. The composition of claim 20, wherein the composition is a lozenge, a powder, a tablet, a gel-cap, a gelatin, a liquid solution, a syrup, an oil, and/or a dissolvable film.

22. The composition of any one of claims 1 to 19, wherein the composition is formulated for administration through injection.

23. The composition of any one of claims 1 to 19, wherein the composition is formulated for topical application and/or intranasal administration.

24. The composition of any of claims 1 to 23, wherein the composition is formulated to decrease inflammation.

25. The composition of any of claims 1 to 24, wherein the composition is formulated to inhibit at least one proinflammatory cytokine.

26. The composition of claim 25, wherein the composition is formulated to inhibit TNF-α and/or IL-6.

27. The composition of any of claims 1 to 26, wherein the composition is formulated to inhibit a prostaglandin.

28. The composition of any of claims 1 to 27, wherein the composition is formulated to inhibit PGE-2.

29. The composition of any of claims 1 to 28, wherein the composition is formulated to treat rheumatoid arthritis.

30. The composition of any of claims 1 to 29, wherein the composition is formulated to prevent rheumatoid arthritis.

31. The composition of any of claims 1 to 30, wherein the composition is formulated to treat polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis.

32. The composition of any of claims 1 to 31, wherein the composition is formulated to prevent polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis.

33. A method of treating a subject at risk for or having any one or more of the following diseases: rheumatoid arthritis, polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis, the method comprising administering any one of the compositions of claims 1 to 32 to the subject, wherein at least one symptom of the disease(s) is ameliorated in the subject and/or the onset of the disease(s) is delayed in comparison to the expected onset of the disease(s) if the patient had not been treated.

34. The method of claim 33, wherein the subject is treated for rheumatoid arthritis or having rheumatoid arthritis, the method comprising administering any one of the compositions of claims 1 to 32 to the subject, wherein at least one symptom of rheumatoid arthritis is ameliorated in the subject and/or the onset of rheumatoid arthritis is delayed in comparison to the expected onset of rheumatoid arthritis if the patient had not been treated.

35. The method of claim 34, wherein the subject is diagnosed as having rheumatoid arthritis.

36. The method of claim 33, wherein the subject is treated for or having any one or more of the following diseases: polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis, the method comprising administering any one of the compositions of claims 1 to 32 to the subject, wherein at least one symptom of the disease(s) is ameliorated in the subject and/or the onset of the disease(s) is delayed in comparison to the expected onset of the disease(s) if the patient had not been treated.

37. The method of claim 34, wherein the subject is diagnosed as having polyarticular juvenile idiopathic arthritis (JIA), psoriatic arthritis (PsA), ankylosing spondylitis (AS), Crohn's disease (CD), hidradenitis suppurativa (HS), ulcerative colitis (UC), chronic plaque psoriasis (Ps), non-infectious intermediate uveitis, non-infectious posterior uveitis, and/or non-infectious panuveitis uveitis.

38. The method of any one of claims 33 to 37, wherein the subject is administered a total amount of between 1 and 10,000 mg, between 10 and 5,000 mg, between 50 and 2,500 mg, or between 100 and 1,000 mg of the biomarker(s) during a 24 hour period.

39. The method of any one of claims 33 to 38, wherein at least one of the biomarker(s) 1 through 22 is synthetically obtained.

40. The method of any one of claims 33 to 39, wherein at least one of the biomarker(s) 1 through 22 is isolated from a plant.

41. The method of claim 40, wherein at least one of the biomarker(s) is isolated from Curcuma longa.

42. The method of any one of claims 33 to 41, wherein the composition has an at least 95% batch-to-batch chemical consistency of relative abundance for the biomarkers.

43. The method of any of claims 33 to 42, wherein the composition further comprises at least one anti-inflammatory drug.

44. The method of claim 43, wherein the at least one anti-inflammatory drug is a nonsteroidal anti-inflammatory drug.

45. The method of claim 44, wherein the nonsteroidal anti-inflammatory drug is acetylsalicylic acid, ibuprofen, ketoprofen, naproxen, one or more of disease-modifying antirheumatic drug (DMARD), salts thereof, or any combination thereof.

46. The method of claim 43, wherein the at least one anti-inflammatory drug is methotrexate, a salt thereof, or any combination thereof.

47. The method of claim 43, wherein the at least one anti-inflammatory drug is a disease-modifying antirheumatic drug (DMARD).

48. The method of claim 47, wherein the DMARD is a biological agent DMARD (biologic DMARD).

49. The method of claim 48, wherein the biologic DMARD is adalimumab, a salt thereof, or any combination thereof.

50. The method of any one of claims 33 to 49, wherein the composition is administered orally.

51. The method of claim 50, wherein the composition is administered as a lozenge, a powder, a tablet, a gel-cap, a gelatin, a liquid solution, a syrup, an oil, and/or a dissolvable film.

52. The method of any one of claims 33 to 49, wherein the composition is administered through injection.

53. The method of any one of claims 33 to 49, wherein the composition is administered topically and/or through intranasal administration.

54. The method of any of claims 33 to 53, wherein a proinflammatory cytokine is inhibited.

55. The method of claim 54, wherein TNF-α and/or IL-6 is inhibited.

56. The method of any of claims 33 to 55, wherein an prostaglandin is inhibited.

57. The method of any of claims 33 to 56, wherein PGE-2 is inhibited.

58. A method of reducing inflammation in a subject, the method comprising administering the composition of any of claims 1 to 32 to a subject, wherein inflammation in the subject is reduced.

59. A method of preventing inflammation in a subject, the method comprising administering the composition of any of claims 1 to 32 to a subject, wherein inflammation in the subject is prevented.

60. A method of inhibiting proinflammatory cytokine production and/or secretion in a subject, the method comprising administering the composition of any of claims 1 to 32 to a subject, wherein the production and/or secretion of a proinflammatory cytokine is reduced.

61. The method of claim 60, wherein the proinflammatory cytokine is TNF-α.

62. The method of any of claims 60 to 61, wherein the proinflammatory cytokine is IL-6.

63. A method of inhibiting prostaglandin production and/or secretion in a subject, the method comprising administering the composition of any of claims 1 to 32 to a subject, wherein the production and/or secretion of a prostaglandin is reduced.

64. The method of claim 63, wherein the prostaglandin is PGE-2.

65. A method of producing a composition of any of claims 1 to 32, wherein the method of producing produces a composition having an at least 90%, preferably at least 95% or at least 98% batch-to-batch chemical consistency of relative abundance for the biomarkers.
